(19) United States
(12) Patent Application Publication
(10) Pub. No.: US 2025/0000689 A1
(43) Pub. Date: Jan. 2, 2025

(54) IMPROVED APPARATUS FOR TREATING SLEEP DISORDERS

(71) Applicants: Vikram Belkhode, Wardha (IN); Sharayu Nimonkar, Wardha (IN)

(72) Inventors: Vikram Belkhode, Wardha (IN); Sharayu Nimonkar, Wardha (IN)

(73) Assignees: Vikram Belkhode, Wardha, MH (IN); Sharayu Nimonkar, Wardha, MH (IN)

(21) Appl. No.: 18/681,655

(22) PCT Filed: Dec. 2, 2021
(86) PCT No.: PCT/IB2021/061225

§ 371 Date: Feb. 6, 2024

(30) Foreign Application Priority Data
Aug. 25, 2021 (IN) 202123038438

(51) Int. Cl. A61F 5/56; A61M 16/04

(52) U.S. Cl. CPC A61F 5/566; A61M 16/0493; A61M 16/0495

(57) ABSTRACT

The present disclosure describes an improved apparatus for treating sleep disorders. The apparatus 100 can include a hollow plate 102, a bulge member 104, and an orifice 106. The hollow plate 102 can be defined by an upper portion 112 and a lower portion 114, with the lower portion 114 connected and being co-extensive with the upper portion 112. Further, the lower portion 114 can be detached/separated from the upper portion 112 whenever desired. The upper portion 112 can be configured to register along a maxillary surface of a subject. The bulge member 104 can be attached about an anterior portion 122 of the hollow plate 102 and is configured to enable resting of tongue of the subject. The orifice 106 can be disposed at an anterior portion 122 of the hollow plate 102 and is configured to allow airflow towards the posterior portion of the hollow plate 102.

### CROSS-REFERENCE TO RELATED APPLICATIONS AND PRIORITY

The present patent application claims priority from Indian Patent Application number 202123038438, filed on Aug. 25, 2021; and PCT WIPO 2023026092 A1 filed on Dec. 2, 2021, the entire contents of both of which are hereby incorporated by reference in their entirety.

### TECHNICAL FIELD

The present invention in general relates to treating sleep disorders. More particularly, the invention relates to an improved apparatus for treating patients suffering from Obstructive Sleep Apnea (OSA).

### BACKGROUND

Background description includes information that may be useful in understanding the present invention. It is not an admission that any of the information provided herein is prior art or relevant to the presently claimed invention, or that any publication specifically or implicitly referenced is prior art.

Obstructive Sleep Apnea (OSA) is an increasingly common disorder that, if left untreated, is associated with an array of adverse health consequences, including cardiovascular disease, metabolic disorders, cognitive impairment, and depression. Obstructive Sleep Apnea (OSA) occurs when the muscles in the back of a patient's throat relaxes more than usual to allow normal breathing. These muscles support structures including the back of the roof of the mouth (soft palate), the triangular piece of tissue hanging from the soft palate (uvula), the tonsils, and the tongue. When the muscles relax, the airway narrows or closes as breathe is taken in and breathing may be inadequate for 10 seconds or longer. This may lower the level of oxygen in the blood and can cause a build-up of carbon dioxide. The brain may sense this impaired breathing and briefly rouses the subject from sleep so that the subject can reopen the airway. This awakening is usually so brief that it may occur that the subject may not remember it. The patient can become awake due to the shortness of breath that corrects itself quickly, within one or two deep breaths. The patient may make a snorting, choking or gasping sound. This pattern can repeat itself five to thirty times or more each hour, all night long. These disruptions can impair the ability to reach the desired deep, restful phases of sleep, and the subject probably may feel sleepy during waking hours. People with obstructive sleep apnea therefore may not be aware that their sleep was interrupted. In fact, many people with this type of sleep apnea think they slept well all night.

Second, OSA is associated with major comorbidities including (i) daytime somnolence; (ii) impaired cognition; (iii) poor quality of life; (iv) increased risk of motor vehicle accidents; (v) there is emerging evidence to suggest that OSA is an independent risk factor for a variety of adverse cardiovascular outcomes; (vi) the clinical disorder, defined as more than five abnormal breathing disturbances (hypopneas or apneas) per hour of sleep combined with symptoms of daytime sleepiness, affects at least 2-4% of the adult population; (vii) although non-obese individuals may suffer from OSA, obesity is the main epidemiologic risk factor; (viii) increases in body mass index; (ix) central accumulation of adipose tissue and neck circumference are strong predictors of disease.

Thus, efforts have been made in the current art to overcome the abovementioned limitations with such efforts although making an attempt at solving leaves open substantial room for improvement. One such solution is the common usage of intraoral device to address OSA. The most commonly used intraoral devices for OSA include (i) mandibular advancement plate or appliance; (ii) mandibular advancement plate with the tongue holder; (iii) palatal lift prosthesis; (iv) electronic vibrating devices. All the aforementioned devices while making an attempt at solving have a tendency to create inconvenience and nuisance in the present anatomy intraoral. Therefore, there exists a need in the art to provide an improved, reliable, and efficient solution for treating patients suffering from obstructive sleep apnea (OSA).

### OBJECTS OF THE PRESENT DISCLOSURE

Some of the objects of the present disclosure, which at least one embodiment herein satisfies are as listed herein below.

It is an object of the present disclosure to provide an apparatus for forming auxiliary airways for treating patients suffering from obstructive sleep apnea.

It is another object of the present disclosure to provide a simple and cost effective apparatus for forming auxiliary airways for treating patients suffering from obstructive sleep apnea.

It is another object of the present disclosure to provide a reliable and efficient apparatus for forming auxiliary airways for treating patients suffering from obstructive sleep apnea.

It is another object of the present disclosure to provide an apparatus that enables ease of cleaning and easy removal of the apparatus.

It is another object of the present disclosure to provide a robust apparatus for forming auxiliary airways for treating patients suffering from obstructive sleep apnea.

It is another object of the present disclosure to provide resting of anterior portion of tongue of the subject within a bulge member for treating patients suffering from obstructive sleep apnea.

### SUMMARY

Before the present apparatuses, methods and systems along with components related thereto are described, it is to be understood that this disclosure is not limited to the particular methods, apparatuses, systems and their arrangement as described, as there can be multiple possible embodiments which are not expressly illustrated in the present disclosure but may still be practicable within the scope of the invention. It is also to be understood that the terminology used in the description is for the purpose of describing the particular versions or embodiments only, and is not intended to limit the scope of the present application. This summary is not intended to identify essential features of the subject matter nor it is intended for use in detecting or limiting the scope of the subject matter.

In one embodiment, an improved apparatus for treating sleep disorders is described. The sleep disorder associated with the subject may be obstructive sleep apnea (OSA). The apparatus may comprise a hollow plate, a bulge member and an orifice. The hollow plate may be defined by an upper portion and a lower portion with the lower portion connected and being co-extensive with the upper portion. The upper portion may be configured to register along a maxillary surface of a subject having a sleep disorder. The lower portion of the hollow plate may be detachable from the upper portion whenever desired by the subject. The bulge member may be attached about an anterior portion of the hollow plate and disposed near anterior portion of the hard palate, enabling anterior portion of tongue of the subject in resting position within the bulge member thus preventing the tongue of the subject to retract towards the mouth region. The orifice may be configured to allow airflow towards the posterior portion of the hollow plate from the anterior portion of the hollow plate. The orifice may be disposed at the anterior portion of the hollow plate

In another embodiment, an improved apparatus for treating sleep disorders is described. The sleep disorder associated with the subject may be obstructive sleep apnea (OSA). The apparatus may comprise a hollow plate, a bulge member and an orifice. The hollow plate may be defined by an upper portion and a lower portion with the lower portion connected and being co-extensive with the upper portion. The upper portion may be configured to register along a maxillary surface of a subject having a sleep disorder. The bulge member may be attached about an anterior portion of the hollow plate and disposed near anterior portion of the hard palate, enabling anterior portion of tongue of the subject in resting position within the bulge member thus preventing the tongue of the subject to retract towards the mouth region. The orifice may be configured to allow airflow towards the posterior portion of the hollow plate from the anterior portion of the hollow plate. The orifice may be disposed at the anterior portion of the hollow plate.

### BRIEF DESCRIPTION OF DRAWINGS

The accompanying drawings are included to provide a further understanding of the present disclosure, and are incorporated in and constitute a part of this specification.

The drawings illustrate exemplary embodiments of the present disclosure and, together with the description, serve to explain the principles of the present disclosure. The diagrams are for illustration only, which thus is not a limitation of the present disclosure.

The detailed description is described with reference to the accompanying Figures. In the Figures, the left-most digit(s) of a reference number identifies the Figure in which the reference number first appears. The same numbers are used throughout the drawings to refer like features and components.

FIG. 1 illustrates an improved apparatus 100 for treating sleeping disorders as implemented on a subject, in accordance to another embodiment of the present disclosure.

FIG. 2 illustrates the improved apparatus 100 for treating sleeping disorders as implemented on a subject with a particular emphasis on efficient airflow associated with the apparatus 100, in accordance to another embodiment of the present disclosure.

### DETAILED DESCRIPTION

Some embodiments of this disclosure, illustrating all its features, will now be discussed in detail. The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

It must also be noted that, the singular forms “a,” “an,” and “the” include plural references unless the context clearly dictates otherwise. Although any methods similar or equivalent to those described herein can be used in the practice or testing of embodiments of the present disclosure, the exemplary methods are now described. The disclosed embodiments are merely exemplary of the disclosure, which may be embodied in various forms.

Various modifications to the embodiment will be readily apparent to those skilled in the art and the generic principles herein may be applied to other embodiments. However, one of ordinary skill in the art will readily recognize that the present disclosure is not intended to be limited to the embodiments illustrated, but is to be accorded the widest scope consistent with the principles and features described herein.

The present disclosure relates to an improved apparatus for treating sleeping disorders. The improved intraoral maxillary device is designed to overcome the problems created by existing intraoral devices. The improved maxillary device/appliance is enables partial detaching/disassembling, easy cleaning, quick replacement, and is comparatively more effective in terms of engagement with the maxillary teeth as compared to existing intraoral devices used for the obstructive sleep apnea.

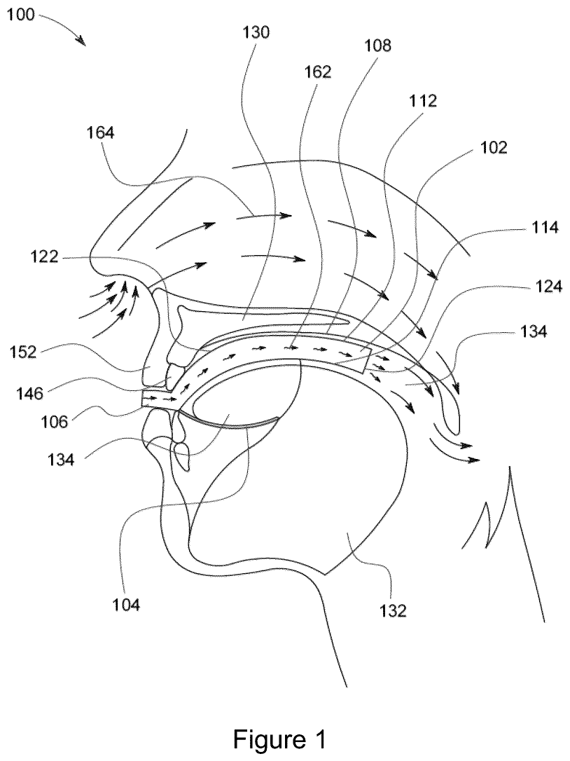

FIG. 1 illustrates an improved apparatus 100 for treating sleeping disorders as implemented on a subject (not shown), in accordance with an embodiment of the present disclosure. The apparatus may comprise a hollow plate 102, a bulge member 104, and an orifice 106. The hollow plate 102 may be defined by an upper portion 112 and a lower portion 114, wherein the lower portion 114 is connected and being co-extensive with the upper portion 112 as can be seen and appreciated. The upper portion 112 may be configured to register along a maxillary surface 108 of a subject (not shown) having a sleep disorder.

In an embodiment, the hollow plate of the apparatus 100 may be formed with two separate parts. The hollow plate may comprise the upper portion 112 and the lower portion 114. The lower portion 114 of the hollow plate can be detached/separated from the upper portion 112 whenever desired by the subject. The upper portion 112 and the lower portion 114 may maintain a constant gap/distance to each other throughout entire length of the apparatus 100. The sleep disorder associated with the subject may be obstructive sleep apnea or any other sleep disorders based on breathing. The bulge member 104 may be attached about an anterior portion 122 of the hollow plate 102 and disposed near anterior portion of the hard palate. The bulge member 104 may enable the anterior portion of tongue of the subject in resting position within the bulge member 104, thus preventing the tongue of the subject to retract towards the mouth region. The bulge member 104 plays a vital role for enabling sufficient delivery of air towards the mouth region, by resting the tongue (in forward position than normal) near the anterior portion 122 of the hollow plate 102. The tongue of the subject may be held/rest in its anatomical and physiologic position via the bulge member 104. In other words, the bulge member 104 may be referred as tongue holding attachment.

In an embodiment, the hollow plate 102 of the apparatus 100 may be formed with two separate parts. The hollow plate may comprise the upper portion 112 and the lower portion 114. The lower portion 114 of the hollow plate can be detached/separated from the upper portion 112 whenever desired by the subject. The lower portion 114 of the hollow plate may be connected to the upper portion 112 via an interlocking means, which are present at the surface of the upper portion 112 and surface of the lower portion 114. The interlocking means may be a mechanical arrangement, but not limited to, screws, dowels, protrusions, and the like.

In another embodiment, the lower portion 114 of the hollow plate may be connected to the upper portion 112 either via an undercut arrangement implemented in the lower portion 114 and the upper portion 112, or via a press fit arrangement. The detachment/separation of the lower portion 114 portion from the upper portion 112 enables easy cleaning of the apparatus whenever the subject desires. Furthermore, the two part hollow plate 102 may provide comfort to the subject along with best adaptability over the subject's maxillary teeth.

In an embodiment, the bulge member 104 may bend downwards and form a shape similar to a scoop. The surface of the bulge member 104 may further comprise a V-shaped notch 150 that facilitates easy entry of the tongue associated with the subject and further enables the provision for the tongue frenulum. Further, the bulge member 104 may prevent the tongue of the subject from slipping out of the bulge member 104, and retract back towards the mouth region.

As further shown in FIG. 1, the orifice 106 may be configured to allow airflow from an anterior portion 122 of the hollow plate 102 towards the posterior portion 124 of the hollow plate 102. The orifice 106 may be disposed at an anterior portion 122 of the hollow plate 102. The orifice 106 may typically allow air to pass through into the interior of the hollow plate 102 through the subject's breathing activity. As can be appreciated, in an embodiment, the placement of the orifice 106 along the hollow plate 102 may depend upon the assessment of an optimal region where airflow intake through the orifice 106 is maximum. As can further be appreciated, the airflow is designed to be directed towards the posterior portion 124 of the hollow plate 102 i.e. a soft palate region 134 of the subject. Thus, in an embodiment, the orifice 106 can be disposed about a central incisor portion of the subject, on the anterior portion 122 of the hollow plate 102. The thickness between the upper portion 112 and the lower portion 114 of the hollow plate 102 is designed such that the airflow through the same is optimal. Various ranges have been identified with regards to the same. Thus, in an embodiment, the thickness associated with the hollow plate 102 between its upper portion 112 and its lower portion 114 is at least 2 mm.

In an embodiment, the apparatus 100 can further include an upper curved plate portion 142 having maxillary teeth receptacle 144 disposed about the anterior portion 122 of the hollow plate 102. The upper curved plate portion 142 of the hollow plate 102 is configured to grasp through its maxillary teeth receptacle 144, at least a portion of maxillary teeth 146 associated with the subject. The maxillary teeth receptacle 144 is designed to provide a support for the apparatus 100 such that the apparatus 100 does not release itself from its implemented position which is as shown in FIG. 1. Thus, in an embodiment, the upper curved plate portion 142 is configured to receive and hold at least an anterior portion of the maxillary teeth 146. In some embodiments, this portion associated with the maxillary teeth 146 can be the central incisor portion of the subject.

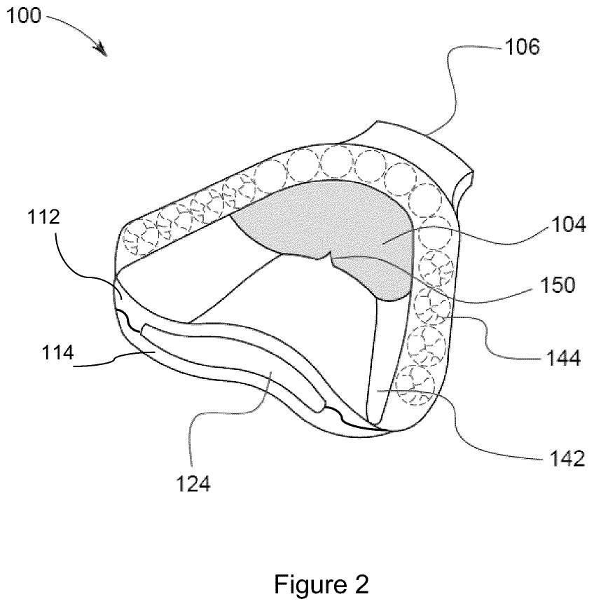

FIG. 2 illustrates an exemplary representation of the apparatus 100 for treating sleeping disorders as implemented on a subject (not shown) with a particular emphasis on efficient airflow associated with the apparatus 100, in accordance with an embodiment of the present disclosure. In the implemented position shown in FIG. 2, the maxillary teeth receptacle 144 is registered with and shown grasping a portion of the maxillary teeth 146. The upper portion 112 of the hollow plate 102 is registered with the maxillary surface 108 while the posterior soft plate member 136 is registered with the soft palate of the subject. The orifice 106 is typically proximate an upper lip 152 of the subject thus having access to fresh air during breathing. The apparatus 100 can typically be used during sleep by the subject. During operation of the apparatus 100, fresh air enters through the orifice 106 and travels through the hollow plate 102 and reaches the posterior portion 124 of the hollow plate 102. The thin arrows 162 can represent the airflow trajectory implemented by the apparatus 100 while the thick arrows 164 can represent the airflow trajectory associated with nasal cavity.

In an embodiment, the apparatus 100 comprising the upper portion 112 and the lower portion 114 may be of Ethyl Vinyl Acetate (EVA) material. At first, the Ethyl Vinyl Acetate material may be placed in a tooth tray. Further, the tooth tray (along with the Ethyl Vinyl Acetate material) may be dipped into warm water in order to soften the Ethyl Vinyl Acetate. In the next step, the tooth tray can be placed in the mouth on upper arch teeth so that the Ethyl Vinyl Acetate can adapt the shape of maxillary teeth of the subject and fit in a perfect manner. Furthermore, the material of the bulge member 104 may be Ethyl Vinyl Acetate (EVA) or any other material having similar properties of flexibility and anti-slipping. In another embodiment, the material of the apparatus 100 may be epoxy resin, wherein the apparatus 100 is fabricated using 3D printing technique It is to be appreciated that the apparatus 100 can be manufactured using any method known in the art including, without limitation, additive manufacturing, injection moulding, etc.

In an embodiment, the apparatus 100 can be custom-made based on the subject. Each and every aspect of the apparatus 100 can be designed based on specific characteristics associated with the subject. For example, for one subject, it may occur that a higher or lower thickness than the aforementioned 2 mm with respect to the hollow plate 102, be relevant and useful. Other factors may include the nature and strength of the maxillary teeth receptacle 144 with respect to its function of grasping the maxillary teeth 146, the nature and strength of the bulge member 104 with respect to resting of the tongue, without limitations. In an embodiment, an apparatus (like 100) can be made based on relevant inputs such as the above by means of computer aided design or CAD.

Thus, the improved maxillary appliance (i.e. the apparatus 100) is an effective intra oral device used for the obstructive sleep apnea. It can also be modified, manipulated (as to its components and its aspects), and utilized for other conditions and disorders. The apparatus 100 is easy to adapt since it fits directly on the patient's teeth. The apparatus 100 can be designed patient to patients. The apparatus 100 helps to maintain contentious air flow through the hole present in anterior region. Due to hollow design of the apparatus 100 the air is directly guided posterior to the tongue thus reducing the obstruction. Since the apparatus 100 remains in proper occlusion with the opposing arch patient feels comfortable to (i) Swallow; (ii) Move the tongue freely in the mouth; (iii) Even drink water with the plate and chew food if required; (iv) Since the vertical height is increased in specific limit that reduce TMJ problems; (v) Assist in respiratory distress such as asthma, SARS/Covid-19, etc.

In accordance with embodiments of the present disclosure, an improved apparatus for treating sleep disorders, described above may have following advantages including but not limited to:
Simple and cost effective apparatus for forming auxiliary airways for treating patients suffering from obstructive sleep apnea.
Reliable and efficient apparatus for forming auxiliary airways for treating patients suffering from obstructive sleep apnea.
Robust apparatus for forming auxiliary airways for treating patients suffering from obstructive sleep apnea.
Improved apparatus for forming auxiliary airways, enabling resting of tongue of the subject within a bulge member for treating patients suffering from obstructive sleep apnea.
Improved apparatus enabling easy cleaning and removal of apparatus (without assistance of other person) whenever desired.
Improved apparatus enabling easy manipulation and is biocompatible.

The embodiments, examples and alternatives of the preceding paragraphs or the description and drawings, including any of their various aspects or respective individual features, may be taken independently or in any combination. Features described in connection with one embodiment are applicable to all embodiments, unless such features are incompatible.

1. An improved apparatus 100 for treating sleep disorders, wherein the sleep disorder associated with the subject is obstructive sleep apnea, the apparatus characterized in that,
a hollow plate 102, defined by an upper portion 112 and a lower portion 114, with the lower portion 114 connected and being co-extensive with the upper portion 112, the upper portion 112 configured to register along a maxillary surface 108 of the subject, wherein the lower portion 114 of the hollow plate is detachable from the upper portion 112 whenever desired by the subject;
a bulge member 104, attached with the lower portion 114, about an anterior portion 122 of the hollow plate 102 and disposed near anterior portion of the hard palate, enabling anterior portion of tongue of the subject in a resting position within the bulge member 104 and preventing the tongue of the subject to retract towards the mouth region; and
an orifice 106, configured to allow airflow towards the posterior portion 124 of the hollow plate 102 from the anterior portion 122 of the hollow plate 102, the orifice 106 disposed at the anterior portion 122 of the hollow plate 102.

2. The apparatus as claimed in claim 1, wherein the orifice 106 is disposed about a central incisor portion of the subject, on the anterior portion 122 of the hollow plate 102.

3. The apparatus as claimed in claim 1, wherein the upper portion 112 and the lower portion 114 of the hollow plate are connected via an interlocking arrangement, present at surface of the upper portion 112 and surface of the lower portion 114.

4. The apparatus as claimed in claim 1, wherein the lower portion 114 of the hollow plate may be connected to the upper portion 112 of the hollow plate either via an undercut arrangement implemented in the lower portion 114 and the upper portion 112, or via a press fit arrangement.

5. The apparatus as claimed in claim 1, wherein the thickness associated with the hollow plate 102 between the upper portion 112 and the lower portion 114 is at least 2 mm.

6. The apparatus as claimed in claim 1, wherein the bulge member 104 comprises a V-shaped notch 150, wherein the notch 150 enables easy entry of the tongue of the subject into the bulge member 104 and resting of the tongue frenulum of the subject.

7. The apparatus as claimed in claim 1, wherein the material of the apparatus 100 and the bulge member 104 is Ethyl Vinyl Acetate.

8. The apparatus as claimed in claim 1, wherein the material of the apparatus 100 is epoxy resin, wherein the apparatus 100 is fabricated using 3D printing technique.

9. The apparatus as claimed in claim 1, wherein the bulge member 104 prevents the slipping out of the tongue of the subject from the bulge member 104, and further avoiding retraction of the tongue towards the mouth region of the subject.

10. The apparatus as claimed in claim 1, wherein the apparatus 100 comprises an upper curved plate portion 142 having a maxillary teeth receptacle 144, disposed about the anterior portion 122 of the hollow plate 102.

11. The apparatus as claimed in claim 10, wherein the upper curved plate portion 142 is configured to receive and hold at least an anterior portion of the maxillary teeth 146.

12. An improved apparatus 100 for treating sleep disorders, wherein the sleep disorder associated with the subject is obstructive sleep apnea, the apparatus characterized in that, comprising:
a hollow plate 102, defined by an upper portion 112 and a lower portion 114 with the lower portion 114 connected and being co-extensive with the upper portion 112, the upper portion 112 configured to register along a maxillary surface 108 of the subject;
a bulge member 104, attached about an anterior portion 122 of the hollow plate 102 and disposed near anterior portion of the hard palate, enabling anterior portion of tongue of the subject in resting position within the bulge member 104 thus preventing the tongue of the subject to retract towards the mouth region; and
an orifice 106, configured to allow airflow towards the posterior portion 124 of the hollow plate 102 from the anterior portion 122 of the hollow plate 102, the orifice 106 disposed at the anterior portion 122 of the hollow plate 102.
